(19) United States
(12) Patent Application Publication
(10) Pub. No.: US 2017/0135628 A1
(43) Pub. Date: May 18, 2017

(54) SMART CONDOM, MANUFACTURING METHOD THEREOF, AND DATA COLLECTION SYSTEM

(71) Applicants: Rulin Chen, Guangzhou (CN); Victor W J Chan, Guangzhou (CN)

(72) Inventors: Rulin Chen, Guangzhou (CN); Victor W J Chan, Guangzhou (CN)

(21) Appl. No.: 15/176,190

(22) Filed: Jun. 8, 2016

(30) Foreign Application Priority Data
Nov. 12, 2015 (CN) 201510776202.X

(51) Int. Cl. A61B 5/00; A61B 5/0205; A61F 6/04

(52) U.S. Cl. CPC A61B 5/486; A61F 6/04; A61B 5/6802; A61B 5/0022; A61B 5/02055; A61B 5/742; A61B 5/01

(57) ABSTRACT

A smart condom comprises a sheath comprising a body (1), an open end (2), a close end (3) and a sensing element (4). A method for making a smart condom comprises mounting a sensing element (4) to a condom before the condom is molded, or during the molding of a condom, or after a condom has been molded, so as to form the smart condom. A system for collecting and analyzing user physical characteristic data and/or user movement data comprises an intelligent terminal and a smart condom having sensing function. With the smart condom and the intelligent terminal of the system, user(s) will be able to directly and accurately get the physical characteristic data and/or movement data in real time, adjust the body condition and control the pace during the sexual intercourse.

### CROSS-REFERENCE TO RELATED APPLICATIONS

The present application claims the benefit of Chinese Patent Application No. 201510776202.X filed on Nov. 12, 2015, the contents of which are hereby incorporated by reference.

### FIELD OF THE INVENTION

The present invention relates to the field of healthcare, more particularly to a smart condom, a method for making a smart condom and a system for collecting and analyzing user physical characteristic data and/or user movement data.

### BACKGROUND OF THE INVENTION

Condoms, also known as prophylactics, are mainly used to prevent pregnancies and sexually transmitted diseases. Since the time it takes to reach orgasm for a male may be different from that for a female sexual partner, conventional condom cannot always satisfy both sexual partners at the same time.

Conventional condoms can't collect or analyze user physical characteristic data or user movement data in real time, as a consequence, the user(s) cannot control the pace during sexual intercourse based on the real-time awareness of the body condition of the user(s). In addition, there has never been a system in the prior art that combines a condom with an intelligent terminal. Therefore, when a user uses a conventional condom during sexual intercourse, the user will be unable to directly and accurately get the data regarding body condition of the user, so that the user or both partners in a sexual activity cannot adjust the body condition to control the pace during the sexual activity based on user physical characteristic data and/or user movement data, as a result, sexual apathy in the female or erectile impotence and premature ejaculation etc. in the male would probably occur which adversely affects sexual harmony.

### SUMMARY OF THE INVENTION

The objective of the instant application is to provide a smart condom that can sense and transmit user physical characteristic data and/or user movement data in real time. The smart condom of the present invention not only protects the user from pregnancies and sexually transmitted diseases, but also monitors the user's body condition in real time so as to help the user control the pace during sexual intercourse.

In order to achieve the above objective, some embodiments of the instant application, in one aspect, provide a smart condom, comprising a sheath, wherein the sheath comprises a body (1), an open end (2), and a closed end (3), characterized in that the sheath is further provided with a sensing element (4).

Preferably, the sensing element (4) is an electronic element configured to sense user physical characteristic data and/or user movement data in real time and transmit the user physical characteristic data and/or the user movement data in real time.

In an embodiment of the instant application, the sensing element (4) comprises a power supply module (41), a sensing module (42), and a data transmission module (43).

The power supply module (41) is configured to provide power to the sensing module (42) and the data transmission module (43) by self-contained power supply or wireless power supply.

The sensing module (42) is configured to sense the user physical characteristic data and/or the user movement data, wherein the user physical characteristic data comprises one or more selected from the group consisting of body temperature, pulse rate, respiration, blood pressure and moisture, and the user movement data comprises one or more selected from the group consisting of velocity, acceleration and pressure.

The data transmission module (43) is configured to transmit data to an intelligent terminal by wireless communication channel.

Preferably, the sensing element (4) is mounted on a surface of the open end (2) of the smart condom or embedded into the open end (2) of the smart condom; or the sensing element (4) is mounted on a surface of the body (1) of the smart condom or embedded into the body (1) of the smart condom; or the sensing element (4) is mounted on a surface of the closed end (3) of the smart condom or embedded into the closed end (3) of the smart condom.

In another aspect, the instant application provides a method for making a smart condom that comprises a sheath comprising a body (1), an open end (2), and a closed end (3), the method comprises the step of: mounting a sensing element (4) to the sheath of the smart condom to form the smart condom.

In one embodiment of the instant application, said mounting the sensing element (4) to the sheath of the smart condom is mounting the sensing element (4) on a surface of a condom mould before the sheath of the smart condom is molded, so as to mold the smart condom.

In another embodiment of the instant application, said mounting the sensing element (4) to the sheath of the smart condom is embedding the sensing element (4) into the sheath of the smart condom during the molding of the sheath of the smart condom, so as to mold the smart condom.

In another embodiment of the instant application, said mounting the sensing element (4) to the sheath of the smart condom is spray printing the sensing element (4) on a surface of the sheath of the smart condom after the sheath of the smart condom has been molded.

Preferably, the method further comprises: mounting the sensing element (4) on a surface of the open end (2) of the smart condom or embedding the sensing element (4) into the open end (2) of the smart condom; or mounting the sensing element (4) on a surface of the body (1) of the smart condom or embedding the sensing element (4) into the body (1) of the smart condom; or mounting the sensing element (4) on a surface of the closed end (3) of the smart condom or embedding the sensing element (4) into the closed end (3) of the smart condom.

In another aspect, the instant application provides a system for collecting and analyzing user physical characteristic data and/or user movement data, characterized in that the system comprises an intelligent terminal and the smart condom. The smart condom is configured to sense the user physical characteristic data and/or the user movement data in real time, and transmit the user physical characteristic data and/or the user movement data to the intelligent terminal in real time. The intelligent terminal is configured to receive the user physical characteristic data and/or the user movement data from the smart condom, process the user physical characteristic data and/or the user movement data in real time, and display the user physical characteristic data and/or the user movement data.

Preferably, the intelligent terminal is also configured to supply wireless power to a power supply module of a sensing element (4) of the smart condom.

The smart condom with sensing function of the instant application is able to collect user physical characteristic data and/or user movement data in real time through the sensing element mounted to the sheath of the smart condom. The user physical characteristic data comprises one or more selected from the group consisting of body temperature, pulse rate, respiration, blood pressure and moisture; the user movement data comprises one or more selected from the group consisting of velocity, acceleration and pressure. The smart condom with sensing function of the instant application is also able to transmit the user physical characteristic data and/or user movement data to the intelligent terminal through wireless communication channel such as Bluetooth, Wi-Fi and RFID (Radio Frequency Identification). Therefore, the user(s) will be able to directly and accurately get the physical characteristic data and/or movement data in real time, adjust the body condition and control the pace during the sexual intercourse.

### BRIEF DESCRIPTION OF THE DRAWINGS

FIG. 1 is a schematic diagram showing a smart condom according to a first embodiment of the instant application.

FIG. 2 is a schematic diagram showing a smart condom according to a second embodiment of the instant application.

FIG. 3 is a schematic diagram showing a smart condom according to a third embodiment of the instant application.

FIG. 4 is a block diagram showing a preferred embodiment of a sensing element of a smart condom of the instant application.

FIG. 5 is a schematic diagram showing a preferred embodiment of a system comprising a smart condom and an intelligent terminal.

FIG. 6 is a schematic diagram showing data interaction between a smart condom and an intelligent terminal of the instant application.

FIG. 7 is a flow chart of making a smart condom according to an embodiment of the instant application.

FIG. 8 is a block diagram showing three ways to mount a sensing element to a sheath of the smart condom.

### DETAILED DESCRIPTION OF ILLUSTRATED EMBODIMENTS

The present invention will be further explained below in detail with reference to figures and particular embodiments.

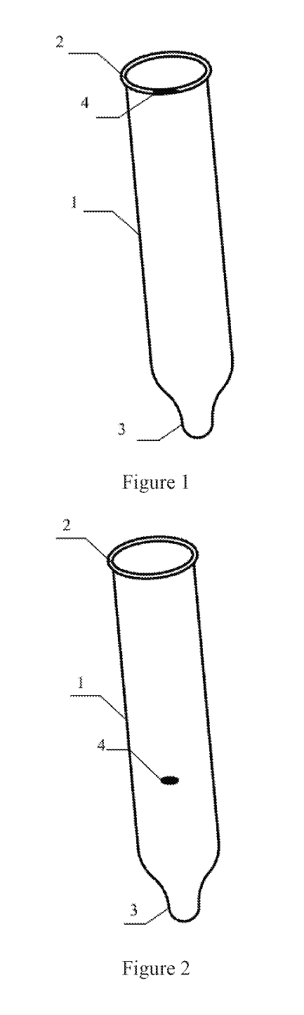

Referring to FIG. 1, FIG. 1 is a schematic diagram showing a smart condom according to a first embodiment of the instant application.

In this embodiment, the smart condom comprises a sheath, the sheath comprises a body 1, an open end 2, a closed end 3, the sheath is further provided with a sensing element 4.

Preferably, the sensing element 4 is an electronic element configured to sense user physical characteristic data and/or user movement data in real time and transmit the user physical characteristic data and/or the user movement data in real time.

In practice, the user physical characteristic data comprises one or more selected from the group consisting of body temperature, pulse rate, respiration, blood pressure and moisture; and the user movement data comprises one or more selected from the group consisting of velocity, acceleration and pressure. Other physical characteristic data and movement data known in the art may also be selected according to actual circumstances.

In this embodiment, as shown in FIG. 1, the sensing element 4 is mounted on a surface of the open end 2 of the smart condom or embedded into the open end 2 of the smart condom. In other embodiments of the instant application, the sensing element 4 can be arranged at other places of the sheath as well.

Referring to FIG. 2, FIG. 2 is a schematic diagram showing a smart condom according to a second embodiment of the instant application. The difference between the first embodiment and the second embodiment is that the sensing element 4 of the second embodiment is mounted on a surface of the body 1 of the smart condom or embedded into the body 1 of the smart condom. The other components of the smart condom or the working principles thereof are the same between the first embodiment and the second embodiment.

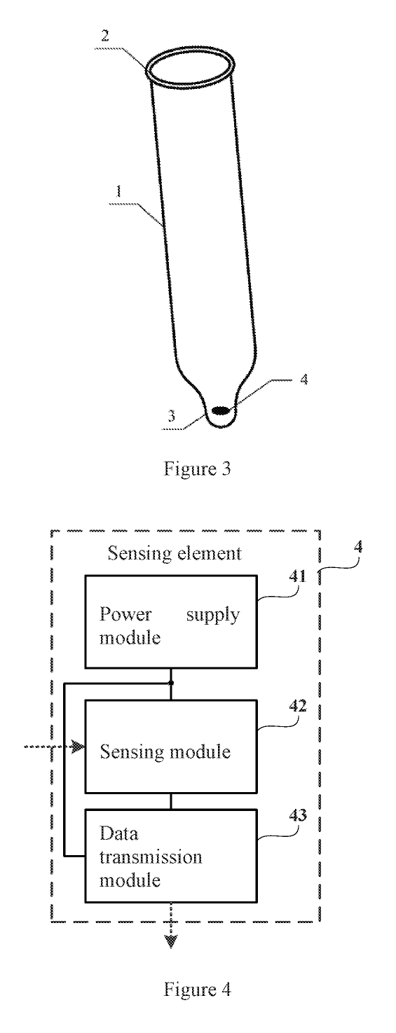

Referring to FIG. 3, FIG. 3 is a schematic diagram showing a smart condom according to a third embodiment of the instant application. The difference between the first embodiment and the third embodiment is that the sensing element 4 is mounted on a surface of the closed end 3 of the smart condom or embedded into the closed end 3 of the smart condom. The other components of the smart condom or the working principles thereof are the same between the first embodiment and the third embodiment.

The smart condom provided by the first, second or third embodiment of the instant application is able to collect user physical characteristic data and/or user movement data through the sensing element mounted to the sheath of the smart condom for further data processing. The user physical characteristic data comprises one or more selected from the group consisting of body temperature, pulse rate, respiration, blood pressure and moisture; and the user movement data comprises one or more selected from the group consisting of velocity, acceleration and pressure.

The sensing element 4 of the smart condom provided by the first, second or third embodiment of the instant application will be described below.

Referring to FIG. 4, FIG. 4 is a block diagram showing a preferred embodiment of a sensing element of a smart condom of the instant application.

The sensing element 4 comprises a power supply module 41, a sensing module 42, and a data transmission module 43.

The power supply module 41 is configured to provide power to the sensing module 42 and the data transmission module 43 by self-contained power supply or wireless power supply.

The sensing module 42 comprises a sensor, the sensing module 42 is configured to sense the user physical characteristic data and/or the user movement data, wherein the user physical characteristic data comprises one or more selected from the group consisting of body temperature, pulse rate, respiration, blood pressure and moisture, and the user movement data comprises one or more selected from the group consisting of velocity, acceleration and pressure.

The data transmission module 43 is configured to transmit data to an intelligent terminal by wireless communication channel such as Bluetooth, Wi-Fi (Wireless Fidelity), RFID (Radio Frequency Identification), ZigBee, and NFC (Near Field Communication).

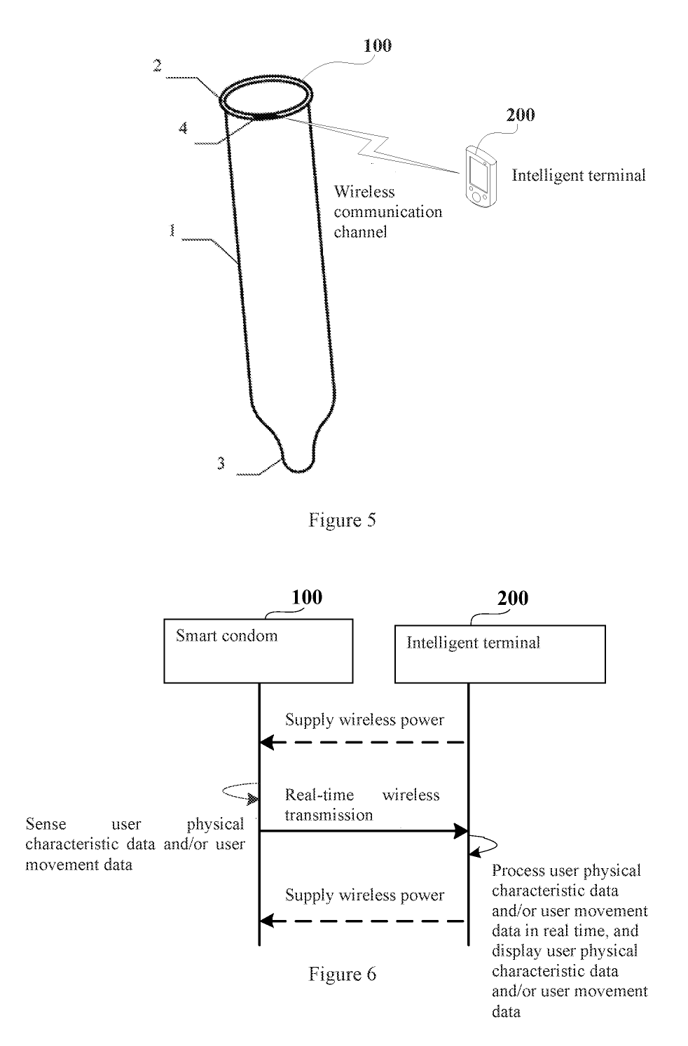

Referring to FIG. 5, FIG. 5 is a schematic diagram showing a preferred embodiment of a system comprising a smart condom and an intelligent terminal, the system collecting and analyzing user physical characteristic data and/or user movement data. Referring to FIG. 6, FIG. 6 is a schematic diagram showing data interaction between a smart condom and an intelligent terminal of the instant application.

The system collecting and analyzing user physical characteristic data and/or user movement data of the preferred embodiment of the instant application is shown in FIG. 5. The system comprises the smart condom 100 with sensing function according to any of the above-described embodiments, and an intelligent terminal 200. The smart condom 100 with sensing function is configured to sense the user physical characteristic data and/or the user movement data in real time, and transmit the user physical characteristic data and/or the user movement data to the intelligent terminal 200 in real time. The intelligent terminal 200 is configured to receive the user physical characteristic data and/or the user movement data from the smart condom 100, process the user physical characteristic data and/or the user movement data in real time, and display the user physical characteristic data and/or the user movement data. In operation, as shown in FIG. 6, the smart condom 100 with sensing function collects the user physical characteristic data and/or the user movement data in real time through the sensing element, and then transmits the user physical characteristic data and/or the user movement data to the intelligent terminal 200 in real time via wireless communication channel; the intelligent terminal 200 thereby receives the user physical characteristic data and/or the user movement data from the smart condom 100, process the user physical characteristic data and/or the user movement data in real time, and display the user physical characteristic data and/or the user movement data. As a result, the user(s) can get aware of his body condition in real time.

Preferably, the intelligent terminal 200 is also configured to supply wireless power to the power supply module 41 of the sensing element 4 of the smart condom. Therefore, the intelligent terminal 200 can provide wireless power to the power supply module 41 of the sensing element 4 of the smart condom before or during the usage of the smart condom 100.

It's noted that FIG. 5 is a schematic diagram that only shows a preferred embodiment of the system in which the sensing element 4 is mounted to the open end 2 of the sheath of the smart condom 100. It should be understood that the sensing element 4 can be mounted to the body 1, the closed end 3 or other parts of the sheath of the smart condom 100 as well, and in these cases, the sensing element 4 transmits data to the intelligent terminal 200 through channels similar to that shown in FIG. 5, for example through wireless communication channel such as Bluetooth, Wi-Fi, RFID, ZigBee, and NFC.

In operation, the intelligent terminal 200 includes but is not limited to smart phones, tablets, portable computers, etc.

The smart condom with sensing function provided by the embodiments of the instant application is able to collect user physical characteristic data and/or user movement data in real time through the sensing element mounted to the sheath of the smart condom. The smart condom with sensing function of the instant application is also able to transmit the user physical characteristic data and/or user movement data to the intelligent terminal through wireless communication channel. Therefore, the user(s) will be able to directly and accurately get the physical characteristic data and/or movement data in real time, adjust the body condition and control the pace during the sexual intercourse.

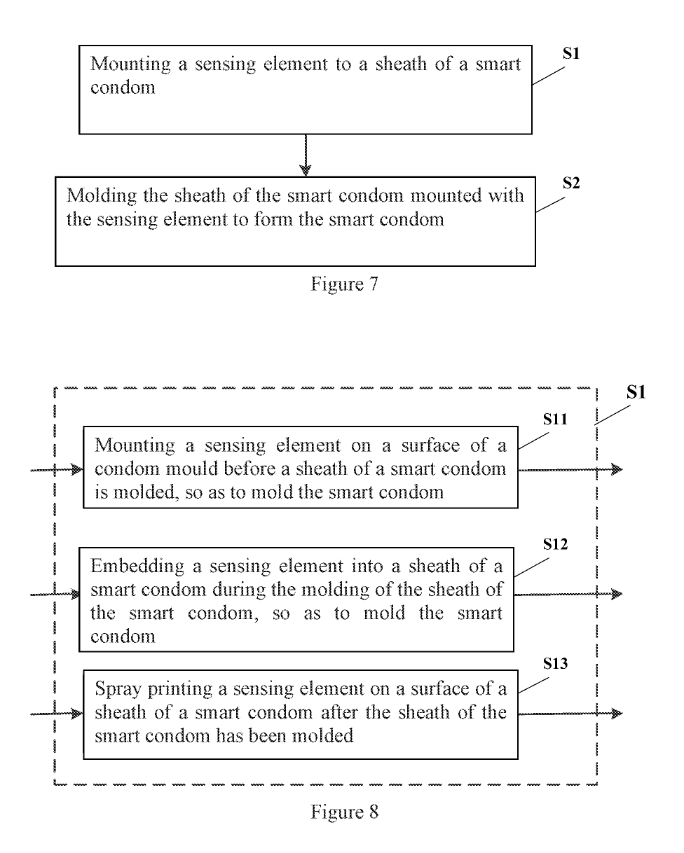

The instant application further provides a method for making a smart condom. Referring to FIG. 7, FIG. 7 is a flow chart of making a smart condom according to an embodiment of the instant application.

In details, a method for making a smart condom that comprises a sheath comprising a body 1, an open end 2, and a closed end 3 comprises the step of:

Step 1: mounting a sensing element 4 to the sheath of the smart condom. Specifically, the sensing element 4 is configured to sense user physical characteristic data and/or user movement data in real time and transmit the user physical characteristic data and/or the user movement data to an intelligent terminal in real time. The sensing element 4 comprises a power supply module 41, a sensing module 42, and a data transmission module 43. The power supply module 41 is configured to provide power to the sensing module 42 and the data transmission module 43 by self-contained power supply or wireless power supply. The sensing module 42 is configured to sense the user physical characteristic data and/or the user movement data, wherein the user physical characteristic data comprises one or more selected from the group consisting of body temperature, pulse rate, respiration, blood pressure and moisture, and the user movement data comprises one or more selected from the group consisting of velocity, acceleration and pressure. The data transmission module 43 is configured to transmit data to an intelligent terminal by wireless communication channel such as Bluetooth, Wi-Fi, RFID, ZigBee, and NFC.

Step 2: forming the smart condom after the sheath of the smart condom mounted with the sensing element 4 has been molded.

In practice, the Step 1 can be realized in several ways. FIG. 8 is a block diagram showing three ways to mount a sensing element to a sheath of the smart condom.

In one embodiment, the Step 1 is Step 11: mounting the sensing element 4 on a surface of a condom mould before the sheath of the smart condom is molded, so as to mold the smart condom.

In another embodiment, the Step 1 is Step 12: embedding the sensing element 4 into the sheath of the smart condom during the molding of the sheath of the smart condom, so as to mold the smart condom

In another embodiment, the Step 1 is Step 13: spray printing the sensing element 4 on a surface of the sheath of the smart condom after the sheath of the smart condom has been molded.

In another embodiment, the method for making the smart condom further comprises:

mounting the sensing element 4 on a surface of the open end 2 of the smart condom or embedding the sensing element 4 into the open end 2 of the smart condom; or

mounting the sensing element 4 on a surface of the body 1 of the smart condom or embedding the sensing element 4 into the body 1 of the smart condom; or

mounting the sensing element 4 on a surface of the closed end 3 of the smart condom or embedding the sensing element 4 into the closed end 3 of the smart condom.

With the help of the sensing element mounted to the sheath of the smart condom, the smart condom with sensing function of the instant application is able to sense and transmit user physical characteristic data and/or user movement data in real time. With a peripheral intelligent terminal, the intelligent terminal receives the user physical characteristic data and/or the user movement data from the smart condom, processes the user physical characteristic data and/or the user movement data in real time, and displays the user physical characteristic data and/or the user movement data. Therefore, the user(s) will be able to directly and accurately get the physical characteristic data and/or the user movement data in real time, adjust the body condition and control the pace during the sexual intercourse.

Although the present invention has been described as above with reference to preferred embodiments, the intention is not to limit the present invention. Those skilled in the art may change or modify the above disclosed technical contents to obtain equivalent embodiments without departing from the scope of the present invention. The present invention is intended to cover all changes, various modifications and equivalent arrangements included within the principle and scope of the present invention according to the technical essence of the present invention.

What is claimed is:

1. A smart condom, comprising a sheath, wherein the sheath comprises a body (1), an open end (2), and a closed end (3), characterized in that the sheath is further provided with a sensing element (4).

2. The smart condom according to claim 1, characterized in that the sensing element (4) is an electronic element configured to sense user physical characteristic data and/or user movement data in real time and transmit the user physical characteristic data and/or the user movement data in real time.

3. The smart condom according to claim 2, characterized in that the sensing element (4) comprises a power supply module (41), a sensing module (42), and a data transmission module (43);
wherein the power supply module (41) is configured to provide power to the sensing module (42) and the data transmission module (43) by self-contained power supply or wireless power supply;
the sensing module (42) is configured to sense the user physical characteristic data and/or the user movement data, wherein the user physical characteristic data comprises one or more selected from the group consisting of body temperature, pulse rate, respiration, blood pressure and moisture, and the user movement data comprises one or more selected from the group consisting of velocity, acceleration and pressure;
the data transmission module (43) is configured to transmit data to an intelligent terminal by wireless communication channel.

4. The smart condom according to claim 3, characterized in that:
the sensing element (4) is mounted on a surface of the open end (2) of the smart condom or embedded into the open end (2) of the smart condom; or
the sensing element (4) is mounted on a surface of the body (1) of the smart condom or embedded into the body (1) of the smart condom; or
the sensing element (4) is mounted on a surface of the closed end (3) of the smart condom or embedded into the closed end (3) of the smart condom.

5. A method for making a smart condom that comprises a sheath comprising a body (1), an open end (2), and a closed end (3), characterized in that the method comprises the step of:
mounting a sensing element (4) to the sheath of the smart condom to form the smart condom.

6. The method according to claim 5, characterized in that said mounting the sensing element (4) to the sheath of the smart condom consists of the step of:
mounting the sensing element (4) on a surface of a condom mould before the sheath of the smart condom is molded, so as to mold the smart condom; or
embedding the sensing element (4) into the sheath of the smart condom during the molding of the sheath of the smart condom, so as to mold the smart condom; or
spray printing the sensing element (4) on a surface of the sheath of the smart condom after the sheath of the smart condom has been molded.

7. The method according to claim 6, characterized in that the method further comprises:
mounting the sensing element (4) on a surface of the open end (2) of the smart condom or embedding the sensing element (4) into the open end (2) of the smart condom; or
mounting the sensing element (4) on a surface of the body (1) of the smart condom or embedding the sensing element (4) into the body (1) of the smart condom; or
mounting the sensing element (4) on a surface of the closed end (3) of the smart condom or embedding the sensing element (4) into the closed end (3) of the smart condom.

8. A system for collecting and analyzing user physical characteristic data and/or user movement data, characterized in that the system comprises an intelligent terminal and the smart condom according to claim 1;
the smart condom is configured to sense the user physical characteristic data and/or the user movement data in real time, and transmit the user physical characteristic data and/or the user movement data to the intelligent terminal in real time;
the intelligent terminal is configured to receive the user physical characteristic data and/or the user movement data from the smart condom, process the user physical characteristic data and/or the user movement data in real time, and display the user physical characteristic data and/or the user movement data.

9. A system for collecting and analyzing user physical characteristic data and/or user movement data, characterized in that the system comprises an intelligent terminal and the smart condom according to claim 2;
the smart condom is configured to sense the user physical characteristic data and/or the user movement data in real time, and transmit the user physical characteristic data and/or the user movement data to the intelligent terminal in real time;
the intelligent terminal is configured to receive the user physical characteristic data and/or the user movement data from the smart condom, process the user physical characteristic data and/or the user movement data in real time, and display the user physical characteristic data and/or the user movement data.

10. A system for collecting and analyzing user physical characteristic data and/or user movement data, characterized in that the system comprises an intelligent terminal and the smart condom according to claim 3;
the smart condom is configured to sense the user physical characteristic data and/or the user movement data in real time, and transmit the user physical characteristic data and/or the user movement data to the intelligent terminal in real time;
the intelligent terminal is configured to receive the user physical characteristic data and/or the user movement data from the smart condom, process the user physical characteristic data and/or the user movement data in real time, and display the user physical characteristic data and/or the user movement data.

11. A system for collecting and analyzing user physical characteristic data and/or user movement data, characterized in that the system comprises an intelligent terminal and the smart condom according to claim 4;
the smart condom is configured to sense the user physical characteristic data and/or the user movement data in real time, and transmit the user physical characteristic data and/or the user movement data to the intelligent terminal in real time;
the intelligent terminal is configured to receive the user physical characteristic data and/or the user movement data from the smart condom, process the user physical characteristic data and/or the user movement data in real time, and display the user physical characteristic data and/or the user movement data.

12. The system according to claim 8, characterized in that the intelligent terminal is also configured to supply wireless power to a power supply module of a sensing element (4) of the smart condom.

13. The system according to claim 9, characterized in that the intelligent terminal is also configured to supply wireless power to a power supply module of a sensing element (4) of the smart condom.

14. The system according to claim 10, characterized in that the intelligent terminal is also configured to supply wireless power to a power supply module of a sensing element (4) of the smart condom.

15. The system according to claim 11, characterized in that the intelligent terminal is also configured to supply wireless power to a power supply module of a sensing element (4) of the smart condom.
